(19) United States
(12) Patent Application Publication
(10) Pub. No.: US 2010/0181197 A1
(43) Pub. Date: Jul. 22, 2010

(54) ISOELECTRIC POINT MARKERS

(75) Inventors: Bengt Bjellqvist, Stockholm (SE); Miles W. Burrrows, Cardiff (GB)

Correspondence Address: GE HEALTHCARE BIO-SCIENCES CORP.;PATENT DEPARTMENT, 101 CARNEGIE CENTER, PRINCETON, NJ, 08540, US

(73) Assignee: GE HEALTHCARE BIO-SCIENCES AB, UPPSALA (SE)

(21) Appl. No.: 12/668,686

(22) PCT Filed: Jul. 14, 2008
(86) PCT No.: PCT/SE08/00449

§ 371 Date: Jan. 12, 2010

(30) Foreign Application Priority Data
Jul. 20, 2007 (GB) 0714202.9

(51) Int. Cl. C07K 1/28; B01D 57/02; G01N 27/447; C09B 23/08; C07K 1/13; C07K 7/06

(52) U.S. Cl. 204/459; 530/330; 530/328; 530/329

(57) ABSTRACT

The present invention relates to the field of isoelectric focussing or 2D electrophoresis, in particular isoelectric point markers with fluorescence detection. The marker comprises an oligopeptide covalently labelled with a fluorescent dye and is characterised in that the dye has a net charge that will maintain the overall net charge of the oligopeptide upon being bound to the oligopeptide. The invention also relates to an electrophoretic method for analysis of proteins using these markers.

### CROSS-REFERENCE TO RELATED APPLICATIONS

This application is a filing under 35 U.S.C. §371 and claims priority to international patent application number PCT/SE2008/000449 filed Jul. 14, 2008, published on Jan. 29, 2009, as WO 2009/014477, which claims priority to patent application number 0714202.9 filed in Great Britain on Jul. 20, 2007.

### FIELD OF THE INVENTION

The present invention relates to the field of isoelectric focussing or 2D electrophoresis, in particular isoelectric point markers with fluorescence detection.

### BACKGROUND OF THE INVENTION

Two-dimensional (2D) electrophoresis remains one of the most frequently applied methods for separating complex mixtures of proteins into their individual components. Whereas one dimensional SDS (sodium dodecyl sulfate) electrophoresis through a cylindrical or slab gel reveals only the major proteins present in a sample, two dimensional polyacrylamide gel electrophoresis (2D PAGE), separates proteins in a first dimension by isoelectric focusing (IEF), i.e., by charge, according to their isoelectric points (pI), and in a second dimension according to size. Thus, 2D PAGE is a more sensitive method of separation and will provide resolution of most of the proteins in a sample. In the isoelectric focusing (IEF) method, a mixture of proteins is placed in a gradient of pH across which an electric field is applied. Proteins may carry either positive or negative charge, or they may be charge neutral, depending on the pH of the environment in which there are placed. In IEF, proteins migrate until they reach a point in the gradient where they carry no net charge, termed the isoelectric point. The isoelectric point of a protein is therefore the pH at which the net charge of the protein is zero. Proteins are positively charged at pH values below their pI and negatively charged at pH values above their pI.

A protein with a positive net charge will migrate toward the cathode, becoming progressively less positively charged as it moves through the pH gradient until it reaches its pI. A protein with a negative net charge will migrate toward the anode, becoming less negatively charged until it also reaches zero net charge. If a protein should diffuse away from its pI, it immediately gains charge and migrates back to its isoelectric position. This is the focusing effect of IEF, which concentrates proteins at their pls and separates proteins with very small charge differences. Because the degree of resolution is determined by electric field strength, IEF is performed at high volt-ages (typically in excess of 1000 V). When the proteins have reached their final positions in the pH gradient, there is very little ionic movement in the system, resulting in a very low final current (typically below 1 mA).

IEF can be run in either a native or a denaturing mode. Native IEF is the more convenient option, as precast native IEF gels are available in a variety of pH gradient ranges. This method is also preferred when native protein is required, as when activity staining is to be employed. The use of native IEF, however, is often limited by the fact that many proteins are not soluble at low ionic strength or have low solubility close to their isoelectric point. In these cases, denaturing IEF is employed.

In order to ensure reliable analytical measurements using IEF, it is essential to employ molecular markers of standards having known pls. Such standards are used in electrophoretic systems for comparison with unknown samples of interest. Among such compounds, ampholytic dyes have been reported based on aminomethylated nitrophenols and aminomethylated sulfophthaleins (Ŝlais, K. et al, J. Chromatography A., (1994), 661, 249-256; Ŝlais, K. et al, J. Chromatography A., (1995), 695, 113-122). Proteins and oligopeptides labelled with coloured or with fluorescent dyes are well known for use as markers in capillary and slab-gel electrophoresis. See for example, WO 02/13848 (Invitrogen Corporation); U.S. Pat. No. 4,356,072 (Saito, H. et al); U.S. Pat. No. 4,107,014 (Suzuki, Y. et al); U.S. Pat. No. 5,866,683 (Shimura, K. et al); Shimura, K. et al, Analytical Chemistry, (2002), 74, 1046-53; Shimura, K. et al, Electrophoresis, (1995), 16, 1479-84; Ŝlais, K. et al, Electrophoresis, (2002), 23, 1682-88. However, markers based on proteins are likely to have many possible reactive sites suitable for conjugation to a fluorescent dye, with the result that heterogeneously labelled pI markers may be produced that are not well focussed under electrophoretic conditions. Synthetic peptides can be prepared with any desired amino acid sequence and have also been shown to be useful pI markers. Such peptides incorporate one or several amino acids with ionic side chains which determine their pI values, as well as a UV absorbing moiety such as tryptophan or a fluorescent label for detection. Provided that each ionic group in the peptide sequence ionizes independently, the pI value of the peptide and the sharpness of its focussing in a pH gradient are reasonably predictable.

The fluorescent dyes which are used to label pI markers are commonly xanthene derivatives, such as fluoresceins and rhodamines. The possibility that such dyes may alter the properties of the native protein upon labelling has been questioned (Bingaman, S. et al, Microcirculation, (2003), 10, 221-231); for example, it has been found that FITC in particular induced significant changes to the physicochemical properties, including pI, of a protein such as BSA. Consequently, there is a need for isoelectric point markers for use in an electrophoretic separation method which overcome the drawbacks of prior art markers as discussed above.

In a first aspect, there is provided an isoelectric point marker for isoelectric focussing or 2D electrophoresis with fluorescence detection comprising an oligopeptide covalently labelled with a fluorescent dye; characterised in that said dye has a net charge that will maintain the overall net charge of the oligopeptide upon being bound to said oligopeptide. In particular, the dye has a net charge that will maintain the charge of the amino acid residue to which the dye is bound.

In another aspect, there is provided a gel electrophoretic separation method for the analysis of proteins, wherein a set comprising two or more different isoelectric point markers are employed each marker in said set comprising an oligopeptide covalently labelled with a fluorescent dye; characterised in that said dye has a net charge that will maintain the overall net charge of the oligopeptide upon being bound to said oligopeptide. Suitably, the isoelectric point pH of each member of the set of oligopeptides has a different value and is suitably in the range of from 3 to 11, more preferably in the range 3.5 to 10.5.

According to the present invention, oligopeptides are disclosed for use as isoelectric point markers, each said oligopeptide being labelled with a fluorescent dye. Suitably, the oligopeptide marker may comprise from about five to about fifteen amino acid residues, preferably from about five to about ten amino acids, and more preferably from about five to about eight amino acid residues. The amino acid sequences which make up the oligopeptide marker are suitably selected from naturally occurring L-amino acids, for example, arginine (Arg or R), asparagine (Asn or N), aspartic acid (Asp or D), glutamine (Gln or Q), glutamic acid (Glu or E), glycine (Gly or G), histidine (H is or H) and lysine (Lys or K). In some examples, additional amino acids such as proline (Pro or P) and methionine (Met or M) may be included in the peptide sequence. It is to be understood that the amino acids making up the oligopeptide marker may also be represented by D-amino acids. Suitably, at least one amino acid making up the peptide sequence is lysine, preferably L-lysine. Preferably, the oligopeptide comprises a carboxy-terminal lysine residue.

In a further embodiment, suitable for 2D electrophoresis, the isoelectric point marker also comprises a group controlling molecular weight/migration position without affecting pI. This group may for example be a polyethylene glycol, PEG, compound.

Particular examples of the isoelectric point markers according to the first aspect are oligopeptides selected from the group consisting of:

| | |
|---|---|
| No. | Sequence |

| | | |
|---|---|---|
| i) | H₂N-Asp-Gly-Asp-Gly-Lys-COOH ------------------------------- | SEQ ID No. 1 |
| ii) | H₂N-Gly-Asp-Gly-Gly-Asp-Gly-Gly-Lys-COOH ------------------- | SEQ ID No. 2 |
| iii) | H₂N-Gly-GlU-Gly-Gly-Glu-Gly-Glu-Lys-COOH ------------------- | SEQ ID No. 3 |
| iv) | H₂N-Gly-Glu-Glu-Gly-Gly-Lys-COOH --------------------------- | SEQ ID No. 4 |
| v) | H₂N-Gly-Gly-Gly-Gly-Glu-Glu-Lys-COOH ----------------------- | SEQ ID No. 5 |
| vi) | H₂N-Glu-Gly-Asp-Gly-Lys-COOH ------------------------------- | SEQ ID No. 6 |
| vii) | H₂N-Glu-Gly-Glu-Gly-Lys-COOH ------------------------------- | SEQ ID No. 7 |
| viii) | H₂N-Gly-Gly-Glu-Gly-Gly-Glu-Gly-Lys-COOH ------------------- | SEQ ID No. 8 |

The examples shown above constitute in toto a set of isoelectric point markers, wherein each said marker is labelled with a fluorescent dye which may be the same or different from the fluorescent dyes employed for labelling the remaining markers in the set. Suitably, the markers disclosed herein are produced by reacting the ω-amino group of a lysine residue with a fluorescent group carrying a positive charge equal to +1. Preferably, the lysine is a C-terminal lysine residue. For peptides with pI-values<7, the lysine group is fully ionised at the isoelectric point. In this pH-range, the lysine will be substituted with a new fully charged positive group. The dipolar character and the zero net charge of the C-terminal group will be maintained. This minimizes the change of pK-values corresponding to the protolytic equilibria of amino acid residues in the vicinity of the reacting C-terminal. The pI-value of the resulting marker will be very similar to the pI-value of the unlabelled oligopeptide. For peptides with pI-values>7, the lysine group cannot be regarded as fully ionised at the isoelectric point and the reaction with a fluorescent group carrying a +1 charge will result in a slight increase of the pI-value of the marker compared with the unlabelled peptide. It is possible to predict the pI-value of the marker from experimental pI-values for comparable peptides and the approximate buffer capacity of the peptides. Alternatively a good approximation will be given from the pI value of the corresponding peptide in which lysine has been substituted with arginine. Preferably, each fluorescent dye label is the same. When the labelled isoelectric point markers are examined together with sample(s) to be studied in an isoelectric gel separation method, each marker can be detected by its fluorescence emission signal and appears as a band in the gel at the position of its isoelectric point.

Alternatively, one or more of the oligopeptides shown above may be combined with additional peptides to form a set of isoelectric point markers within the range of pI 3 to pI 11, more preferably in the range pI 3.5 to pI 10.5, each of said peptides being covalently labelled with a fluorescent dye such that the overall net charge of the oligopeptide upon being labelled is maintained. Further examples of oligopeptides suitable for use as pI markers may be selected from the following:

| | |
|---|---|
| No. | Sequence |

| | | |
|---|---|---|
| ix) | H₂N-Gly-His-Glu-Gly-Glu-Gly-Lys-COOH -------------------- | SEQ ID No. 9 |
| x) | H₂N-Gly-His-Gly-His-Gly-Glu-Gly-Glu-Gly-Lys-COOH -------- | SEQ ID No. 10 |
| xi) | H₂N-Gly-Gly-His-Gly-Gly-Glu-Gly-Lys-COOH ---------------- | SEQ ID No. 11 |
| xii) | H₂N-Met-Gly-Lys-Gly-Glu-Lys-COOH ------------------------ | SEQ ID No. 12 |
| xiii) | H₂N-Gly-Gly-Lys-Gly-Glu-Lys-COOH ------------------------ | SEQ ID No. 13 |
| xiv) | H₂N-Pro-Gly-Lys-Gly-Glu-Lys-COOH ------------------------ | SEQ ID No. 14 |
| xv) | H₂N-Gly-Tyr-Lys-Tyr-Gly-Lys-COOH ------------------------ | SEQ ID No. 15 |
| xvi) | H₂N-Gly-Tyr-Lys-Gly-Lys-COOH ---------------------------- | SEQ ID No. 16 |

The amino acid sequences of the isoelectric point markers were selected essentially as described in Shimura, K. et al, Electrophoresis, (2000), 21, 603-610. The markers contain a minimum of two proteolytic groups with pK values in the vicinity of the pI value of the corresponding marker. The pH scale is a pH scale defined for isoelectric focussing in 8M urea and 20° C. as described in Bjellqvist, B. et al, Electrophoresis, (1993), 14, 1357-1365, Bjellqvist, B. et al, Electrophoresis, (1994), 15, 529-539.

Current methods for the prediction of the pI-values for proteins and peptides are based on different degrees of simplification. Shimura et al (loc cit) uses the same pK-value for amino acid residues participating in reactions with water (Asp, Glu, His, Cys, Tyr, Lys and Arg) irrespective of whether the residues appear terminally or internally in the peptide. The pK-values for the C-terminal carboxylic groups are all identical (pK=3.6) independent of the nature of the C-terminal amino acid; pK-values for all N-terminal amino groups are similarly set to 7.6. Bjellqvist et al (loc cit) give different pK-values for the Asp, Glu, His, Cys, Tyr, Lys and Arg residues, depending on whether they appear as internal, C-terminal or N-terminal residues and also give slightly different values to the C-terminal carboxyl and the N-terminal amino group depending on the nature of the C- and N-terminal group respectively. The latter is the one used on the Expasy server.

Both methods are based on the equilibria of amino acid residues participating in reactions with water and can be described with one pK-value for each type of residue, which is independent of the nature of neighbouring amino acids. The consequence of this assumption is that for large groups of tryptic peptides, the same pI-value will be predicted. FIG. 1 shows a plot of experimental pI-values for 630 tryptic peptides which, in addition to an N-terminal lysine and an N-terminal amino group, contain two internal glutamic acid residues as the only charged amino acid residues. The approaches described above will result in the same pI-value for all these peptides. The value predicted at the Expasy server is 4.53, which can be compared with the value resulting from the experimental distribution, i.e. 4.49±0.075.

In reality, the presence of a charged group in the vicinity of an amino acid residue participating in a protolytic equilibrium will also effect the pK-value of the equilibrium and, if the pK-value is in the vicinity of the pI-value of the peptide, also the pI-value of the peptide. This is shown in FIG. 2, in which the 630 tryptic peptides have been divided into subgroups based on the distance between the positively charged N-terminal and the closest glutamic acid residue. As can be seen from this figure, a short distance between the N-terminal and the closest glutamic acid residue decreases the pI-values of the peptides and it is evident that it is possible to make more precise pI predictions (±0.03-0.04 pH units) when the relative positions of charged groups are taken into account. Generally, the presence of a positively charged group in the vicinity of acidic amino acid residues containing either a carboxylic group (Asp or D, Glu or E, or the C-terminal), a sulfhydryl group (Cys or C) or an acidic hydroxyl group (Tyr or Y) will stabilize these residues in their basic negatively charged forms. This corresponds to a decrease in the pK-values of the reactions involved and results in a decrease in pI, provided that the pK-values is not too far removed from the pI-value. For basic amino acid residues (His or H, Lys or K, Arg or R and the N-terminal), the effect on the pK-values will be reversed. The presence of negatively charged groups in the vicinity of a basic amino acid will stabilize the residue in the positively charged form, thereby resulting in an increase of the pK-value. For reasons of symmetry, the pK-decrease induced by a positively charged basic amino acid residue on the pK-value describing the equilibrium of a neighbouring acidic amino acid residue will correspond to an equally large pK-increase of the pK-value of the basic residue induced by the neighbouring negatively charged acidic amino acid residue.

It would be expected that the presence of two negatively charged amino acid residues at limited distances from each other along the amino acid chain should result in an increase of the pK-values involved and a concomitant increase of the pI-value, while two positively charged residues at limited distances from each other along the peptide chain could be expected to give reversed effect, i.e. a decrease in pK-values and a concomitant decrease of the pI-value. In reality, no such effects can be detected. Table 1 shows the average pI-values found by varying the distance between the two glutamic acid residues in peptides having two such glutamic acid residues as the only charged internal amino acids, no glutamic acid in positions 1-6 and a minimum of one amino acid residue between the C-terminal and closest glutamic acid residue. In the peptides, C-terminal amino acids are either lysine or arginine. As can be seen from the Table, the distance between the glutamic acid residues has a negligible effect on resulting pI-values. This is explained by the presence of two oppositely charged amino acid residues in the chain, thereby favouring configurations with a short distance between the two residues. As a consequence, pK-values are affected also when these residues are separated by up to 5 amino acid residues. On the other hand, the presence of two equally charged residues favours configurations with the longest possible distance between the charged groups and for neighbouring charged residues, the average distance becomes too long to generate a detectable effect on pK and pI values.

TABLE 1
Dependence of experimentally determined pI-values in an IPG strip pH
4.25-4.65 on the distance between two glutamic acid residues

| Number of amino acid | | |
|---|---|---|
| residues between the glutamic | | |
| acid residues | Average pI-value | Number of peptides |
| 0 | 4.538 ± 0.035 | 66 |
| 1 | 4.558 ± 0.030 | 42 |
| 2 | 4.542 ± 0.030 | 35 |
| 3 | 4.548 ± 0.037 | 14 |
| 4 | 4.523 ± 0.035 | 8 |

The C-terminal, which in tryptic digests is either lysine or arginine contains both a positively charged group and a negatively charged group, gives notable effects on resulting pI-values only when the neighbouring group is charged. At larger distances, for peptides having two internal glutamic acid residues (no glutamic acid in position 1-6), the C-terminal amino acid being either lysine or arginine, the effect on pI is negligible (Table 2).

TABLE 2
Dependence of experimentally determined pI-values in an IPG strip pH
4.25-4.65 on the distance between the C-terminal and closest
glutamic acid residue

| Number of amino acid residues | | |
|---|---|---|
| between the C-terminal and | | |
| closest glutamic acid residue | Average pI-value | Number of peptides |
| 0 | 4.588 ± 0.031 | 77 |
| 1 | 4.539 ± 0.032 | 34 |
| 2 | 4.543 ± 0.034 | 64 |

For aspartic acid and glutamic acid residues, the presence of a positively charged group comprising a histidine, a lysine, an arginine or an N-terminal amino group, 1-5 amino acid residues away from an aspartic or glutamic acid residue will influence the pK-values of the reactions involving these acidic groups and as a consequence the pI-values of the peptides involved. Positively charged groups are expected to have a similar influence on the pK-values of negatively charged C-terminal having a carboxylic acid group (cysteine and tyrosine residues). A corresponding effect is expected on histidine, lysine and arginine residues, as well as on N-terminal amino groups from negatively charged groups that occur between 1-5 amino acid residues away. For the C-terminal groups, lysine and arginine, which in addition to the carboxylic group also contain a positively charged group, interaction influencing the pI value is only detected when the charged group is the closest neighbour to the C-terminal lysine or arginine residue. Similar short range effects extending only to the closest neighbour can be expected also for histidine at C-terminal, as well as for N-terminal glutamic acid and aspartic acid which contain a positively charged group and a negatively charged group. Other effects that neighbouring amino acids have on pK-values and pI-values appear to be negligible. The results show that groups of tryptic peptides will have approximately the same pI-values (distributions corresponding to standard deviations of the order ±0.03-0.04 pH-units) provided that:

a) all peptides in the group contain the same type and number of chargeable groups;

b) the distance between the different types of residues carrying oppositely charged group either is larger than 5 alternatively is the same for all peptides in the group; and

c) when the peptides possess charged groups as the closest neighbour to either a glutamic acid or aspartic acid N-terminal, or alternatively, charged groups are the closest neighbour to a C-terminal lysine or arginine, all peptides in the group should have the same charged group as the closest neighbour to the corresponding N- or C-terminal.

According to the present invention, a fluorescent dye is employed in the labelling reaction, the dye having a net charge that will maintain the overall net charge of the oligopeptide upon being bound to the oligopeptide. Thus, the dye is able to compensate for charge lost at the amino acid residue participating in the labelling reaction, thereby minimizing the change of the pK value describing the protolytic equilibria of amino acid residues in the vicinity of the reacting residue. Without being bound by theory, it is to be expected that the pI-value of the resulting labelled pI-marker should be similar to the pI of the unlabelled peptide, provided that the charge of the group consumed in the labelling reaction and the charge of the group added for charge compensation have the same net charge at the pI-value. It should be possible to predict from experimental pI-values, the pI-value of the labelled peptide, provided that the conditions described in conditions a), b), and c) above are met.

Table 3 shows the experimental pI-values determined for a series of acidic pI markers, reacted at a C-terminal lysine group with CY™ 5. These values are compared with: i) the pI-values predicted for the unlabelled peptides assuming that the pK-values are unaffected by neighbouring amino acids calculated at the Expasy server and ii) the average experimental pI-values found for this set of peptides. As can be seen from the Table, the approach normally used for pI prediction gives approximately correct pI-values, but the difference between predicted pI-value and the pI-value of the pI-marker can be as large as 0.3-0.4 pH-units. If the pI-value of the marker is predicted instead from determined experimental pI-values for comparable peptides, the result is in excellent agreement with experimentally determined pI-values. An initial tryptic digestion is frequently used as an alternative to 2D electrophoresis in connection with biomarker detection and evaluation. The digestion is followed by separation, identification and quantification of resulting peptides. Isoelectric focusing is increasingly used as one of the separation steps prior to MS based identification. As a consequence, the number of tryptic peptides with experimentally determined pI-values has increased considerably. There is therefore, basis for precise prediction of the pI-value resulting from a tryptic peptide having lysine as a C-terminal amino acid, and further for a pI marker, where the ω-amino group of lysine is coupled to a group containing a positive charge, thereby compensating for the effects of the charged amino group.

TABLE 3

| | | | | | | |
|---|---|---|---|---|---|---|
| | | | | | Average pI- | |
| | | | Sequence of | | value _{(ecp)} for | |
| | | pI | Comparable | No. of | Comparable | pI (exp) |
| No. | Sequence | (calc) | Peptides | Peptides | Peptides | pI/marker |

| | | | | | | |
|---|---|---|---|---|---|---|
| xvii) | H₂N-Asp-Gly-Asp- | 4.21 | Asp-X-Asp- | 15 | 3.800 ± 0.024 | 3.86 |
| | Gly-Lys(CY™5)- | | Xₙ-Lys alt | | | |
| | COOH | | Arg | | | |
| | (SEQ ID NO: 1) | | | | | |
| xviii) | H₂N-Gly-Asp-Gly- | 4.21 | X-Asp-X- | 86 | 3.977 ± 0.041 | 4.02 |
| | Gly-Asp-Gly-Gly- | | Xₙ-Asp-Xₙ- | | | |
| | Lys(CY™5)-COOH | | Lys alt Arg | | | |
| | (SEQ ID NO: 2) | | | | | |
| xix) | H₂N-Gly-Glu-Gly- | 4.25 | X-Glu-X- | 15 | 4.226 ± 0.011 | 4.22 |
| | Gly-Glu-Gly-Glu- | | Xₙ-Glu-Xₙ- | | | |
| | Lys(CY™5)-COOH | | Glu-X-Lys | | | |
| | (SEQ ID NO: 3) | | alt Arg | | | |
| xx) | H₂N-Gly-Glu-Glu- | 4.53 | X-Glu-Glu- | 15 | 4.361 ± 0.020 | 4.44 |
| | Gly-Gly- | | Xₙ-Lys | | | |
| | Lys(CY™5)-COOH | | | | | |
| | (SEQ ID NO: 4) | | | | | |
| xxi) | H₂N-Gly-Gly-Gly- | 4.53 | X-X-X-X- | 17 | 4.528 ± 0.023 | 4.56 |
| | Gly-Glu-Glu- | | Glu-X-Glu- | | | |
| | Lys(CY™5)-COOH | | Lys alt Arg | | | |
| | (SEQ ID NO: 5) | | | | | |
| xxii) | H₂N-Glu-Gly-Asp- | 4.37 | Glu-X-Asp- | 4 | 4.168 | 4.18 |
| | Gly-Lys(CY™5)- | | Xₙ-Lys alt | | | |
| | COOH | | Arg | | | |
| | (SEQ ID NO: 6) | | | | | |
| xxiii) | H₂N-Glu-Gly-Glu- | 4.53 | Glu X-Glu- | 12 | 4.303 ± 0.018 | 4.28 |
| | Gly-Lys(CY™5)- | | Xₙ-Lys alt | | | |
| | COOH | Arg | | | | |
| | (SEQ ID NO: 7) | | | | | |
| xxiv) | H₂N-Gly-Gly-Glu- | 4.53 | X-X-Glu-X- | 107 | 44.33 ± 0.041 | 4.42 |
| | Gly-Gly-Glu-Gly- | | Xₙ-Glu-Xₙ- | | | |
| | Lys(CY™5)-COOH | | Lys alt Arg | | | |
| | (SEQ ID NO: 8) | | | | | |

X = any amino acid residue except Asp, Glu, His, Lys, Arg; n = number of consecutive X-residues with n ≧ 1

The expectation that the pI-value of the marker and the pI of the comparable peptides should be identical is only valid for pI-values lower than ˜7. At higher pH values the differences between the positive charge of the lysine amino group and the charge of the CY™ 5 group results in a pI-difference, which increases with pH.

Table 4 shows the predicted pI for unlabelled peptides having lysine or arginine as C-terminal (calculated at the Expasy server) and the predicted pI-values of corresponding labelled pI markers with the charge of +1 at the C-terminal. It can be seen from Table 4 that for pH>7, the difference between an unlabelled peptide and the corresponding marker falls in the region 0.15-0.3 pH-units. The arginine residue is a much stronger base than lysine and, as a result, peptides having arginine as C-terminal will have pI-values which more closely agree with the pI-values of the labelled markers and which according to Table 4 should be useful for precise prediction of the values for the pI-markers. As an alternative, knowledge of the experimental pI for a comparable peptide with lysine as C-terminal will, when combined with an estimated buffer capacity of the peptide allow pI prediction for a pI-marker with a precision better than ±0.1 pH-unit.

TABLE 4

Unlabelled Peptide

| | | | | |
|---|---|---|---|---|
| | | | Lys as | Arg as |
| No. | Sequence | pI-Marker* | C-terminal | C-terminal |

| | | | | | |
|---|---|---|---|---|---|
| xxv) | H₂N-Gly-His-Glu-Gly-Glu-Gly- | SEQ ID No: 9 | 5.40 | 5.40 | 5.40 |
| | Lys(CY™5)-COOH ---------------- | | | | |
| | --- | | | | |
| xxvi) | H₂N-Gly-His-Gly-His-Gly-Glu-Gly- | SEQ ID No: 10 | 6 | 6 | 6 |
| | Glu-Gly-Lys(CY™5)-COOH ---- | | | | |
| xxvii) | H₂N-Gly-Gly-His-Gly-Gly-Glu-Gly- | SEQ ID No: 11 | 6.75 | 6.75 | 6.75 |
| | Lys(CY™5)-COOH --------------- | | | | |
| xxviii) | H₂N-Met-Gly-Lys-Gly-Glu- | SEQ ID No: 12 | 8.5 | 8.35 | 8.5 |
| | Lys(CY™5)-COOH ---------------- | | | | |
| | ------- | | | | |
| xxix) | H₂N-Gly-Gly-Lys-Gly-Glu- | SEQ ID No: 13 | 8.75 | 8.59 | 8.75 |
| | Lys(CY™5)-COOH ---------------- | | | | |
| | -------- | | | | |
| xxx) | H₂N-Pro-Gly-Lys-Gly-Glu- | SEQ ID No: 14 | 9.18 | 9.01 | 9.18 |
| | Lys(CY™5)-COOH ---------------- | | | | |
| | -------- | | | | |
| xxxi) | H₂N-Gly-Tyr-Lys-Tyr-Gly- | SEQ ID No: 15 | 9.70 | 9.53 | 9.70 |
| | Lys(CY™5)-COOH ---------------- | | | | |
| | -------- | | | | |
| xxxii) | H₂N-Gly-Tyr-Lys-Gly-Lys(CY™5)- | SEQ ID No 16 | 9.99 | 9.70 | 10.00 |
| | COOH ----------------------- | | | | |

*predicted from Expasy server.

The selection, firstly of the attachment site and secondly, the choice of a particular dye suitable for conjugation to the oligopeptide isoelectric point marker is important. Lysine, cysteine, aspartic acid and glutamic acid each carry a functional group that is capable of modification; however, the primary ω-amino group of lysine is particularly useful because of its reactivity towards acylation reagents. Thus, in the present invention, the fluorescent dye is preferably covalently coupled to the oligopeptide via a single lysine residue present in the oligopeptide, where preferably the lysine is located at the carboxy-terminus of the peptide. As described herein, it is important to compensate for any gain or loss in charge upon reaction of the unlabelled peptide with a labelling reagent since isoelectric focusing depends on charge. The chemistry of labelling biomolecules with fluorescent dyes is well documented. See for example, Garman, A. J., Non-radioactive Labelling: A Practical Introduction, Academic Press (1999). Preferred acylation reagents are those which employ a stable activated ester of a carboxylic acid such as N-hydroxysuccinimidyl-, or N-hydroxysulfosuccinimidyl- esters of carboxylic acid derivatised dyes. Other groups reactive towards primary amino functions are selected from isothiocyanate, isocyanate, acid halide and acid anhydride.

Fluorescent dyes suitable for labelling the isoelectric point markers described herein are dyes which carry a net +1 charge. Particularly suitable dyes are the fluorescent cyanine dyes (Mujumdar, R. B. et al., Cytometry, (1989), 10, 11-19; Waggoner et al., U.S. Pat. No. 5,268,486). The cyanine dyes have the following general structure (I):

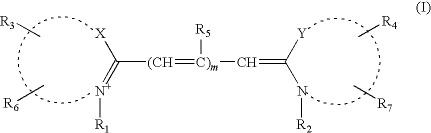

where X and Y is selected from O, S or >C(CH₃)₂, m is an integer from 1 to 3 and at least one of R₁, R₂, R₃, R₄, R₅, R₆ or R₇ is a reactive group which reacts with amino, hydroxy or sulfhydryl nucleophiles. The dotted lines represent carbon atoms necessary for the formation of the cyanine dye, preferably forming on ring or two fused rings having 5 or 6 atoms in each ring. The reactive group can be any known reactive group, which in the context of the present invention is covalently reactive with a primary amino group of the oligopeptide. Suitably, the reactive group R₁, R₂, R₃, R₄, R₅, R₆ or R₇ is attached to the dye chromophore either directly or via a linker group (L) and includes reactive moieties such as groups containing isothiocyanate, acid halide, N-hydroxysuccinimidyl ester and N-hydroxysulfosuccinimidyl ester.

Preferably, the cyanines attach to the peptide via the activated ester of an alkanoic acid linker, more preferably hexanoic acid. Although conjugation of the dye to the oligopeptide destroys the charge of the lysine side chain, the intrinsic positive charge in the dye compensates, thus maintaining the overall charge within the isoelectric point marker. In the cyanine dye molecule, two functionalized indole rings are connected via a polymethine linker chain. The spectral characteristics of the cyanine dyes can be easily modulated by changing the length of the linker between the indole rings of the dye. A longer or shorter polymethine linker will result in different absorption and fluorescence emission wavelengths and thus, different colours emitted by the dye. Preferably each of the oligopeptides is labelled with a dye having the same molecular mass as the remaining oligopeptides in the set.

Suitably, the fluorescent dye employed for labelling an isoelectric point marker according to the present invention is a cyanine dye having the structure (II):

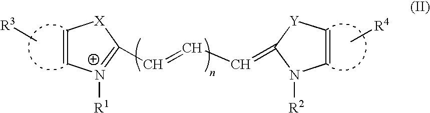

wherein the dotted lines each represent carbon atoms necessary for the formation of one or two fused rings having six carbon atoms in each ring; X and Y are selected from the group consisting of S, O and CH₃—C—CH₃;
n is an integer selected from 1, 2 or 3;
one of R¹ and R² is a group reactive with a nucleophilic group and the remaining R¹ or R² is an alkyl, or is a group containing one or two positive charges; and
R³ and R⁴ are selected from hydrogen and sulfonic acid.

Preferably, X and Y in the above formula are the same and are CH₃—C—CH₃. Preferably n=1 or 2, more preferably n=2. Thus, particularly advantageous dyes are pentamethine cyanine dyes, for example Cy™ 5

Particular examples of fluorescent cyanine dyes for useful for labelling the isoelectric point markers of the present invention are represented by compounds of formula (III) and (IV).

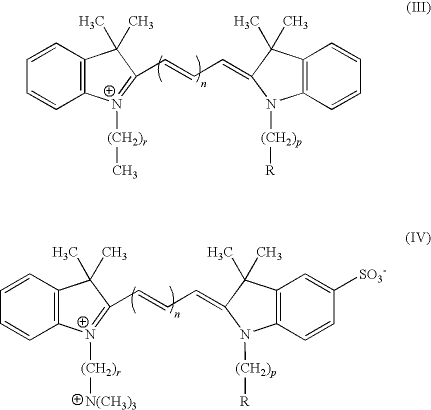

In structures (III) and (IV), R is selected from the group consisting of isothiocyanate, acid halide, N-hydroxysuccinimidyl ester and N-hydroxysulfosuccinimidyl ester; n is an integer selected from 1, 2 or 3, r is 1 or 2 and p is an integer from 1 to 5, preferably 5.

Preferably, R is N-hydroxysuccinimidyl ester or N-hydroxysulfosuccinimidyl ester. As described hereinbefore, the number of methine groups linking the heterocyclic ring systems defines the absorption maxima and fluorescence emission of the dyes. Thus, the fluorescence emission peaks of a CY™ 3 dye (n=1) is 570 nm (red), while the fluorescence emission of a CY™ 5 dye (n=2) is 670 nm (far red). As described above, O or S or a combination thereof can be placed in the X and Y positions in place of >C(CH₃)₂.

Preferred CY™ 5-labelled isoelectric point markers are selected from the following:

| | |
|---|---|
| No. | Sequence |

| | | |
|---|---|---|
| xvii) | H₂N-Asp-Gly-Asp-Gly-Lys(CY™5)-COOH ----------- | SEQ ID No. 1 |
| | --- | |
| xviii) | H₂N-Gly-Asp-Gly-Gly-Asp-Gly-Gly- | SEQ ID No. 2 |
| | Lys(CY™5)-COOH -- | |
| xix) | H₂N-Gly-Glu-Gly-Gly-Glu-Gly-Glu-Lys(CY™5)-COOH | SEQ ID No. 3 |
| | -- | |
| xx) | H₂N-Gly-Glu-Glu-Gly-Gly-Lys(CY™5)-COOH ------- | SEQ ID No. 4 |
| | ---- | |
| xxi) | H₂N-Gly-Gly-Gly-Gly-Glu-Glu-Lys(CY™5)-COOH --- | SEQ ID No. 5 |
| | --- | |
| xxii) | H₂N-Glu-Gly-Asp-Gly-Lys(CY™5)-COOH ----------- | SEQ ID No. 6 |
| | --- | |
| xxiii) | H₂N-Glu-Gly-Glu-Gly-Lys(CY™5)-COOH ----------- | SEQ ID No. 7 |
| | --- | |
| xxiv) | H₂N-Gly-Gly-Glu-Gly-Gly-Glu-Gly-Lys(CY™5)-COOH | SEQ ID No. 8 |
| | -- | |

Alternative dyes may be used in place of the cyanines, such as dipyrromethine boron difluoride dyes, the derivatized 4,4-difluoro-4-bora-3a,4a,-diaza-S-indacene dyes, described in U.S. Pat. No. 4,774,339 (Haugland et al.) which are sold by Molecular Probes, Inc. under the trademark BODIPY®. The BODIPY® dyes, which have no net charge, can be covalently linked to lysine side chains using an activated N-hydroxysuccinimidyl ester which forms an amide bond. The result is the loss of the lysine positive charge. Therefore, a single positively charged linker group must be used in order to replace the lost charge of the primary amine group of the lysine. Procedures for making BODIPY® dyes are described in U.S. Pat. No. 4,774,339. Incorporation into the dye moiety of a positively charged linker group can be effected by techniques well known to those skilled in the art. Alternately, dyes that modify other amino acid residues may be used, provided the ionic and charge characteristics of the amino acid are preserved by the modification. For example, the attachment site on the oligopeptide may be a sulfhydryl or carboxylic group. When a sulfhydryl group is the attachment site on the oligopeptide, the corresponding attachment site on the dye is an iodoalkyl or a maleimido group. When a carboxylic acid group is the attachment site on the oligopeptide, the corresponding attachment site on the dye is a carbodiimide.

Procedures for preparing peptides of defined sequence are well known. See for example, Methods in Enzymology, Volume 289, “Solid-Phase Peptide Synthesis” Ed. Gregg B. Fields, Academic Press 1997. Automated peptide synthesis may be performed on an Applied Biosystems 433A peptide synthesizer using a piperidine-labile 9-fluorenylmethoxy-carbonyl (Fmoc) coupling strategy. Wang resin is employed as solid phase support. Typically, the following coupling conditions are applied to synthesise the peptide. To the resin (0.25 mmol), is added a four fold excess of the required amino acid (1 mmol) in N-methylpyrrolidone (NMP) followed by 1 mmol of coupling reagent O-benzotriazol-1-yl-N,N,N′,N′-tetramethyluronium hexafluorophosphate/1-hydroxybenzotriazole (HBTU/HOBt, 0.1M/0.45M) in dimethylformamide (DMF) and diisopropylethylamine (DIPEA) (2M, 1 ml). Deprotection of Fmoc residues is achieved using a 22% piperidine solution in NMP and monitored via a conductivity trace for completion. All amino acids are protected using standard side chain protecting groups compatible with the Fmoc strategy (tBu/BOC). Following solid phase synthesis, the resin-bound peptide can be isolated in near quantitative yield. The simultaneous removal of side-chain protecting groups and cleavage of the peptide from the resin may be carried out with trifluoroacetic acid (TFA) containing 2.5% triisopropylsilane (TIS) and 2.5% water (2 ml) for one hour. Precipitation of the peptide is induced by dropwise addition to diethyl ether. The peptide is then further washed in ether and collected via centrifugation to yield crude product, which can be further purified by preparative RP-HPLC and lyophilised for storage.

For preparative HPLC, an ÄKTAEXPLORE™ instrument may be used with a VYDAC® C18 21.5×250 mm protein and peptide column, eluent A being 0.1% TFA/Water and eluent B acetonitrile. Analytical HPLC can be performed on an ÄKTAEXPLORE™ instrument using a VYDAC® C18 4.6×250 mm protein and peptide column employing the same eluent as in the preparative method.

The N-terminus remains Fmoc protected following cleavage from the resin in order to facilitate the orthogonal labelling of the lysine ω-amino group.

The pI markers described herein may be used as a control for determining that an isoelectric focusing experiment is performing in the correct manner. Furthermore, the determined pI value of each marker enables convenient and accurate calibration of protein components in a sample to be studied. In a typical example, the marker is applied with the sample on an IPG strip to be used as first dimension in 2-D electrophoresis. If CY™ 5-tagged isoelectric point markers are employed, samples may be labelled with CY™ 3 and/or CY™ 2. Alternatively, post-staining methods for detection may be used. Following electrophoresis in the first dimension, focusing the strips can be scanned prior to the equilibration step preceding the second dimension run. Verification that the first dimension run has worked is of great value in 2-D electrophoresis as the most common reasons to failure relate to the first dimension runs, while most of the work in 2-D experiments relates to the second dimension run (equilibration, gel casting, staining and evaluation).

The pI markers may also be used to determine experimental pI-values with good precision. Depending on the context, the determined values are used in different ways. One possibility is to compare the experimental value with the pH-value predicted from the composition of protein as given from protein databases such as SWISS-PROT, EMBL or PIR. In connection with the identification of peptides from MS/MS-spectra with the aid of programs as SEQUEST and Mascot comparison of experimental and predicted pI-values has been used as a tool to validate true identifications and exclude false positives. See for example, Holmes, M. et al, Anal. Chem., (2004), 76(2), 276-282; Cargile, B. J. et al, J. Biomol. Tech., (2005), 16(3), 181-189; Cargile, B. J. et al, J. Proteome Res., (2004), 3(1), 112-9. It is also possible to use database information to calculate the net-charge of protein or peptide at the experimental isoelectric point. If the result approaches one charge unit, this strongly indicates that the database information utilized is either incorrect or incomplete (Bjellqvist, B. et al, Electrophoresis, (1993), 14, 1357-1365, Bjellqvist, B. et al, Electrophoresis, (1994), 15, 529-539).

Data on pI-values of tryptic peptides will enable selection of peptide markers with pI-values optimal for a certain applications or alternatively, for use with specific techniques. Irrespective of whether the pH-gradient to be used for focusing is a linear or non-linear pH gradient, it will be possible to select peptides which will result in pI marker mixture with the focusing positions evenly distributed along the resulting pH-gradient. The high precision with which the pI of the markers can be predicted will allow synthesis of marker mixture suitable also for specified very narrow pH gradients spanning 0.3-1 pH units.

Isoelectric point markers may also be designed with pI-values very close to the focusing position of a protein or peptide of interest. This is useful in situations where there is a need to continue analysis of the focused entity over time. One example is peptide focusing, where there could be a need to extract the focussed peptide from a polyacrylamide gel for subsequent qualitative and/or quantitative analysis with LC-MS/MS.

In use, the labelled isoelectric point markers described herein overcome the drawbacks of conventional markers, particularly with regard to their use at low pH range e.g. pH 4. The present markers employ environmentally insensitive cyanine dyes. By contrast, markers labelled with xanthine dyes such as the fluoresceins and rhodamines have a carboxylic acid group which has a pKa of approximately 4. This means that near to pH 4 the charge on the dye will change and therefore cause perturbation of the overall marker pI.

### BRIEF DESCRIPTION OF THE DRAWINGS

The invention is further illustrated by reference to the following examples and figures in which:

FIG. 1 shows a plot of experimental pI-values for 630 tryptic peptides which, in addition to an N-terminal lysine and an N-terminal amino group, contain two internal glutamic acid residues as the only charged amino acid residues;

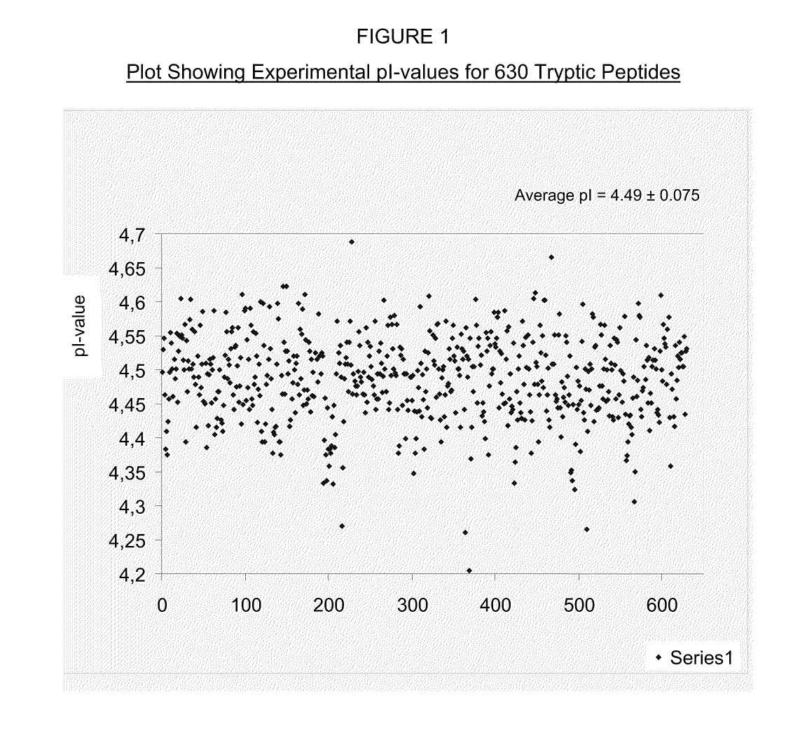

FIG. 2 illustrates the effect on pI of the distance between the positively charged N-terminal and the closest glutamic acid residue;

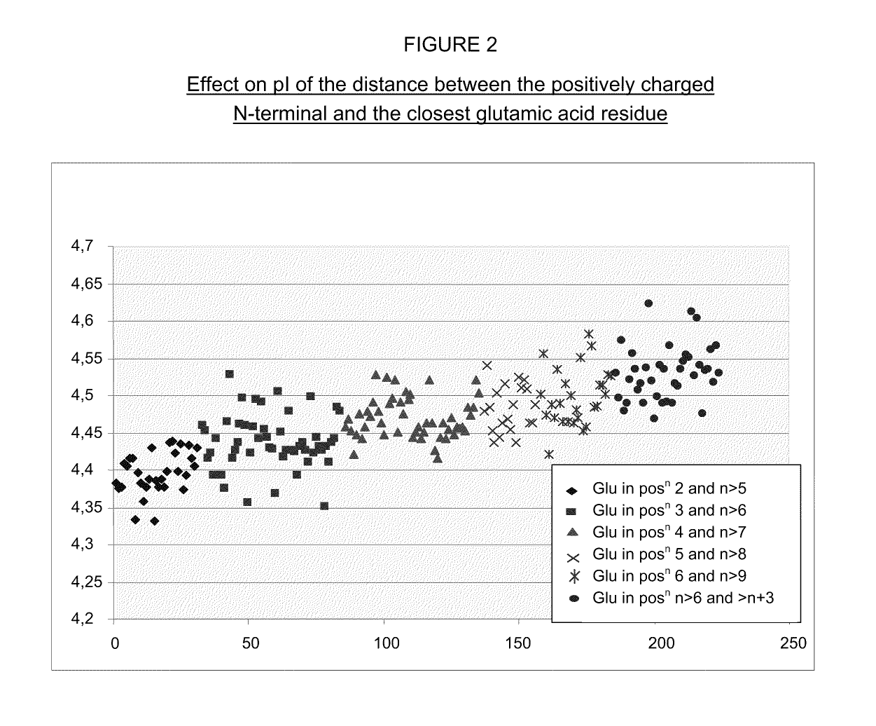

FIG. 3 shows the pI-marker H₂N-Asp-Gly-Asp-Gly-Lys(CY™ 5)—COOH (Compound xvii, SEQ ID No: 1) focused in linear IPG pH 3.70-4.05. (0.3 μg/strip) for 129 kVh;

FIG. 4 shows the focusing positions of pI-markers in a non-linear pH gradient pH 3.4-4.9.

### EXAMPLES

The present example is provided for illustrative purposes only, and should not be construed as limiting the present invention as defined by the appended claims.

### 1. Preparation of H₂N-Glu-Gly-Asp-Gly-Lys(CY™ 5)—COOH (Compound xxii, SEQ ID No: 6))

### 1.1H₂N-Glu-Gly-Asp-Gly-Lys-COOH (Compound No. vi SEQ ID No: 6)

Peptide synthesis was undertaken on an Applied Biosystems 433A peptide synthesizer using a 0.25 mmol 9-fluorenylmethoxy-carbonyl (Fmoc) coupling strategy. A four fold stoichiometric excess (1.0 mmol) of each of the following amino acid residues was used; N-α-Fmoc-L-glutamic acid α-t-butyl ester, N-α-Fmoc-L-glycine, N-α-Fmoc-L-aspartic acid α-t-butyl ester and N-α-Fmoc-N-ε-t-butyloxycarbonyl-L-lysine. Wang resin (0.25 mmol, 309 mg, 0.81 mmol/g) was used as solid phase support.

To the resin (0.25 mmol), was added four fold excess amino acid (1 mmol) in N-methylpyrrolidone (NMP) followed by 1 mmol of coupling reagent O-benzotriazol-1-yl-N,N,N′,N′-tetramethyluronium hexafluorophosphate/1-hydroxybenzotriazole (HBTU/HOBt, 0.1M/0.45M) in dimethylformamide (DMF) and diisopropylethylamine (DIPEA) (2M, 1 ml). Deprotection of Fmoc residues was achieved using a 22% piperidine solution in NMP and monitored via a conductivity trace for completion. The N-terminal amine remained Fmoc protected post synthesis to facilitate the orthogonal labelling of the lysine amine.

Following solid phase automated synthesis, the resin bound peptide (100 mg) was added to a solution (2 ml) of trifluoroacetic acid (TFA) containing 2.5% triisopropylsilane (TIS) and 2.5% water, and allowed to roll at ambient temperature for one hour. Precipitation of the peptide was attempted by the dropwise addition of the cleavage solution through a frit to diethyl ether (35 ml). As no precipitation was observed in this case, solvent was removed in vacuo to leave a thin film which, upon washing with ethyl acetate (35 ml), afforded a white solid. The peptide was taken up into a 10% acetonitrile/water solution (5 ml) and purified via reverse phase high performance liquid chromatography using an AKTAEXPLORE™ instrument with a VYDAC® C18 21.5×250 mm protein and peptide column. A gradient method of 25-40% B over 35 minutes was employed. Eluant A was 0.1% TFA/Water and eluant B was acetonitrile. Following lyophilisation, 35 mg of peptide was isolated. MALDI MS (α-cyano-4-hydroxycinnamic acid matrix) [M+H]⁺ion of 727.6 observed, consistent with that of the desired product.

### 1.2 Fluorescence Labelling: H₂N-Glu-Gly-Asp-Gly-Lys (CY™ 5)—COOH (SEQ ID No: 6)

The peptide from 1.1 (35 mg, 0.048 mmol) was placed in a glass dimple vial, to which was added CY™ 5 NHS ester (20 mg, 0.034 mmol), dimethylformamide (3 ml) and diisopropylethylamine (150 μl). The vial was sealed and allowed to roll at ambient temperature for 1.5 hours in the absence of light. Following the reaction period, the solution was added to cold diethyl ether (35 ml) to afford precipitation of the labelled peptide sequence. The blue solid was collected by centrifugation and further washed with another aliquot of diethyl ether (35 ml). Purification of the labelled peptide was achieved via high performance liquid chromatography using an ÄKTAEXPLORE™ instrument with a VYDAC® C18 21.5×250 mm protein and peptide column. The sample was injected in a 10% acetonitrile/water solution (5 ml), eluent A was 0.1% TFA/Water and eluent B was acetonitrile. A gradient of 25 to 95% B over a period of 30 minutes was used. Following lyophilisation, 410 ug of material was isolated. MALDI MS (α-cyano-4-hydroxycinnamic acid matrix) [M+H]⁺ion 969.3 observed, consistent with that of the desired product. UV/Vis spectroscopy λ max 641 nm.

### 2. The following peptides were synthesised and fluorescently labelled with CY™ 5 by methods analogous to that described in Example 1

| | | [M + H]⁺ |
|---|---|---|
| No. | Sequence | Fmoc-peptide-OH |
| i) | H₂N-Asp-Gly-Asp-Gly-Lys-COOH | 714 |
| | (SEQ ID No: 1) | |
| ii) | H₂N-Gly-Asp-Gly-Gly-Asp-Gly-Gly-Lys-COOH | 885 |
| | (SEQ ID No: 2) | |
| iii) | H₂N-Gly-Glu-Gly-Gly-Glu-Gly-Glu-Lys-COOH | 984 |
| | (SEQ ID No: 3) | |
| iv) | H₂N-Gly-Glu-Glu-Gly-Gly-Lys-COOH | 798 |
| | (SEQ ID No: 4) | |
| v) | H₂N-Gly-Gly-Gly-Gly-Glu-Glu-Lys-COOH | 855 |
| | (SEQ ID No: 5) | |
| vii) | H₂N-Glu-Gly-Glu-Gly-Lys-COOH | 742 |
| | (SEQ ID No: 7) | |
| viii) | H₂N-Gly-Gly-Glu-Gly-Gly-Glu-Gly-Lys-COOH | 912 |
| | (SEQ ID No: 8) | |

### 3. Focusing of pI-marker H₂N-Asp-Gly-Asp-Gly-Lys(CY™ 5)—COOH (SEQ ID No: 1) in a 22 cm long IPG strips pH 3.70-4.05

3.1 Yeast proteins (Saccharomyces cerevisiae, type II (1 gram) were dissolved in a 10 ml solution containing 50 mM Tris-HCl pH 8.0, 6 M urea and 10 mM DDT. The solution was allowed to stand for 1 hour at 37° C. and then allowed to react with iodoacetamide (addition of 250 μl 0.8 M iodoacetamide solution) for 30 minutes at room temperature to acylate the cysteine groups. The sample was then sonicated 5 times for 30 seconds followed by dilution of the sample in 50 mM Tris-HCl, 1 mM CaCl₂ (1 ml sample to 9 ml Tris-Cl buffer solution). 2.5 ml of diluted sample was added to a container with 250 μg trypsin (4 containers for 10 ml diluted sample) and digestion was allowed to proceed for 2 hours at 37° C. The sample was then concentrated by evaporation to 2 ml. 1 ml of digested concentrated sample was then added to a NAP-10 column pre-equilibrated with 15 ml 8 M urea solution and the sample was eluted with 1.5 ml urea. After a protein determination the sample was aliquoted and frozen until used.

3.2 Sample application was performed with polyacrylamide gel pieces, cast as 1 mm thick gel of the size 260×110 mm on a GelBond PAGE film. In addition to acrylamide (38.8 gram/litre) and bisacrylamide (1.2 gram/litre) the gel polymerising solution contained 15.0 mM Immobiline pK 3.6 and 5.9 mM Immobiline pK 9.3. The polymerisation initiators used were ammonium persulfate and tetramethyl ethylenediamine. The gel was extensively washed in water (10 wash cycles of 20 minutes using 200 ml water/wash). To the last wash was added 2% glycerol. The gel was then dried and cut to generate a number sample application gel pieces of the size 43×7 mm.

Four, 22 cm long IPG strips were re-swollen overnight in 250 μl 8 M urea solution containing 1% Pharmalyte pH 2.5-5 and a trace of bromophenol blue. In parallel, four sample application gel pieces were re-swollen in 315 μl sample solution containing 300 μg yeast protein in 8 M urea to which 0.3 μg of the pI marker had been added.

The four re-swollen IPG strips were positioned in a ceramic manifold in the IPGPHOR™ 3 with the GelBond film downwards. At the basic/cathodic ends of the IPG strips, paper electrode wicks (10×7×1 mm) to which 150 ul water had been added, were positioned on top of the IPG strips with an overlap of 2 mm to establish electric contact. At the anodic end, the wetted electrode wick was positioned 39 mm away from the acidic end of the IPG strip. The re-swollen acidic sample application gel was positioned with the gel side down as a bridge between the paper electrode wick and the IPG strip to give contact both with the paper wick and with the IPG strip through a 2 mm overlap region. The anodic and cathodic electrodes were finally positioned on the anodic and cathodic paper electrode wicks respectively and a light pressure was applied on the back of the sample application gels at the over lap regions with the paper electrode wicks and the IPG strips respectively.

3.3 Focusing was then performed at 20° C. with maximum current set to 50 uA/strip with the following voltage settings:

| | | Voltage (volts) | Time (hrs:mins) |
|---|---|---|---|
| 1. | gradient | 0-500 | 0:01 |
| 2. | gradient | 500-4000 | 3:00 |
| 3. | gradient | 4000-6000 | 3:00 |
| 4. | gradient | 6000-10000 | 3:00 |
| 5. | step | 10000 | 90:00 |

The focusing experiment was performed with current limitations from 1000V onwards; 2500 V was reached after 5 hours and the voltage only changed slowly to reach a final voltage of 2715 V after 52 hours 23 minutes and a total volthourproduct of 129 kilovolthours.

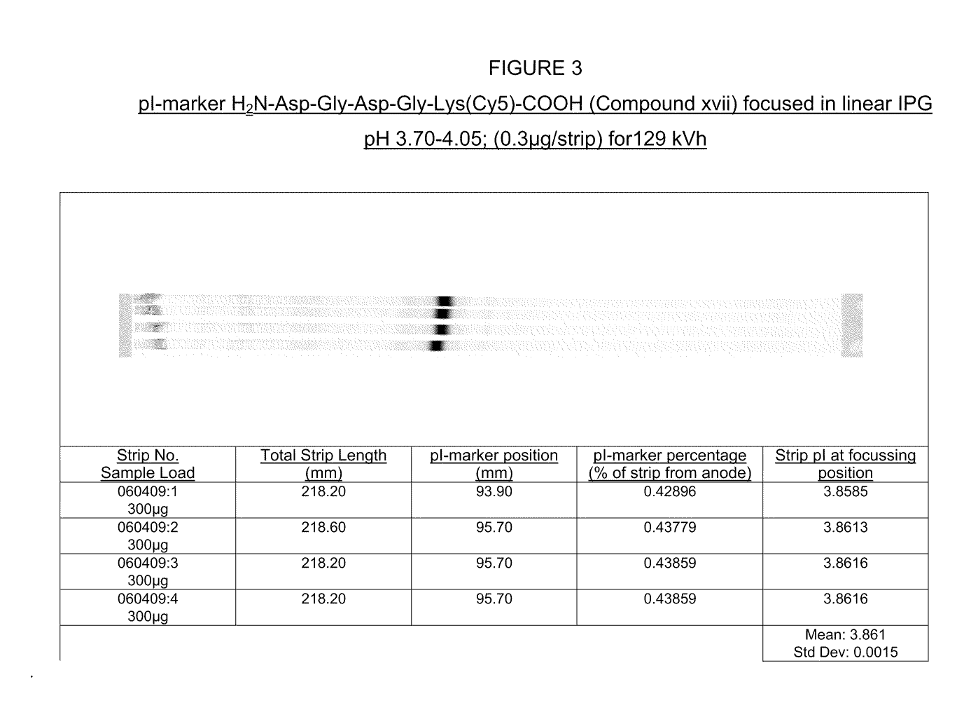

Upon completion, the gel strips were transferred to a TYPHOON™ 9400 imager and scanned in the fluorescence mode at 633 nm (FIG. 3). From the focusing positions the pI-value for the marker was determined to be 3.86.

A total of 6612 tryptic yeast peptides have been identified after focusing in IPG strips 3.7-4.05. Sixteen of these peptides have a structure (Table 5) which should give them a pI in close agreement with the pI of the marker. The average value found for these peptides 3.80±0.03 is in reasonable agreement with the value found for the pI marker.

TABLE 5
pI-vaIues determined for 16 tryptic peptides *

| | |
|---|---|
| Sequence | pI-value |

| | |
|---|---|
| Asp-Cys-Asp-Ser-Leu-Ser-Ser-Leu-Val-Thr-Arg | 3.765 |
| (SEQ ID NO: 17) | |
| Asp-Thr-Asp-Tyr-Val-Pro-Ile-Val-Val-Val-Gly-Asn-Lys | 3.840 |
| (SEQ ID NO: 18) | |
| Asp-Ser-Asp-Val-Thr-Trp-Leu-Tyr-Gly-Pro-Ile-Val-Arg | 3.800 |
| (SEQ ID NO: 19) | |
| Asp-Leu-Asp-Leu-Pro-Thr-Gln-Gln-Ile-Leu-Val-Ala-Arg | 3.830 |
| (SEQ ID NO: 20) | |
| Asp-Leu-Asp-Ala-Val-Asn-Gly-Ser-Asn-Gly-Ser-Lys | 3.790 |
| (SEQ ID NO: 21) | |
| Asp-Gln-Asp-Ser-Ala-Val-Val-Ser-Ser-Asn-Ile-Lys | 3.823 |
| (SEQ ID NO: 22) | |
| Asp-Thr-Asp-Met-Val-Leu-Ile-Pro-Ala-Gly-Val-Pro-Arg | 3.808 |
| (SEQ ID NO: 23) | |
| Asp-Ile-Asp-Ile-Leu-Val-Asn-Asn-Ala-Gly-Lys | 3.828 |
| (SEQ ID NO: 24) | |
| Asp-Gly-Asp-Tyr-Asn-Leu-Val-Gly-Ser-Lys | 3.746 |
| (SEQ ID NO: 25) | |
| Asp-Ile-Asp-Asn-Gly-Tyr-Thr-Leu-Ser-Leu-Met-Tyr-Lys | 3.800 |
| (SEQ ID NO: 26) | |
| Asp-Gly-Asp-Gln-Val-Val-Phe-Met-Val-Ser-Gln-Lys | 3.760 |
| (SEQ ID NO: 27) | |
| Asp-Asn-Asp-Val-Gln-Leu-Ala-Ala-Thr-Lys | 3.800 |
| (SEQ ID NO: 28) | |
| Asp-Val-Asp-Leu-Val-Asn-Ala-Ala-Arg | 3.800 |
| (SEQ ID NO: 29) | |
| Asp-Ala-Asp-Leu-Leu-Asn-Ser-Val-Val-Val-Gln-Arg | 3.775 |
| (SEQ ID NO: 30) | |
| Asp-Leu-Asp-Ala-Ala-Gln-Gly-Thr-Leu-Lys | 3.815 |
| (SEQ ID NO: 31) | |
| Asp-Ser-Asp-Tyr-Ile-Pro-Val-Val-Val-Val-Gly-Asn-Lys | 3.825 |
| (SEQ ID NO: 32) | |
| Average pI: | 3.80 |
| Standard Deviation | 0.027 |

* K or R as C-terminal and 0 in position 1 and 3. No other amino acid residues with charged side chains and at least one uncharged residue between position 3 and the C-terminal amino acid.

### 4. Focusing of the pI-markers: H₂N-Asp-Gly-Asp-Gly-Lys(CY™ 5)—COOH (SEQ ID NO: 1), H₂N-Glu-Gly-Asp-Gly-Lys (CY™ 5)—COOH (SEQ ID NO: 6), H₂N-Glu-Gly-Glu-Gly-Lys(CY™ 5)—COOH (SEQ ID NO: 7), and H₂N-Gly-Gly-Gly-Gly-Glu-Glu-Lys(CY™ 5)—COOH (SEQ ID NO: 5), in 22 cm long IPG strips pH 3.70-4.05.

Focusing was performed in IPG strips pH 3.4-4.9 containing a non-linear pH-gradient adjusted to give an even distribution of peptides in the focused strip. The experiment was run essentially as described in Example 3. For sample application, the same type of polyacrylamide gel applicator was used, this time re-swollen in an 8M urea solution containing 30 μg of the digested yeast proteins and 0.3 μg of each of the four pI-markers. Focusing was continued for 47 hours 45 minutes and the total volthourproduct was 207 kVh.

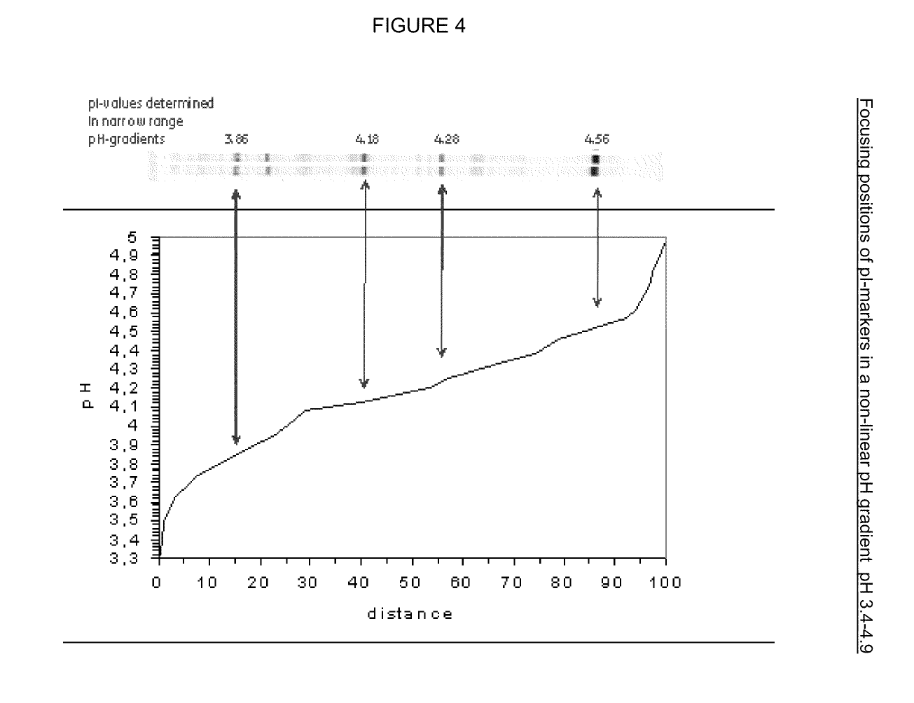

With a current limitation of 50 μA/strip a final voltage of 4550 V was reached. The image resulting from the fluorescence scan is shown in FIG. 4 and compared with the expected pH gradient for the IPG strip, pH 3.4-4.9. The focussing positions found for the markers show that the pH gradient in the strip is 0.02-0.04 pH units more acidic than expected.

It is to be understood that any feature described in relation to any one embodiment may be used alone, or in combination with other features described, and may also be used in combination with one or more features of any other of the embodiments, or any combination of any other of the embodiments. Furthermore, equivalents and modifications not described above may also be employed without departing from the scope of the invention, which is defined in the accompanying claims.

1. An isoelectric point marker for isoelectric focussing or 2D electrophoresis with fluorescence detection comprising an oligopeptide covalently labelled with a fluorescent dye, wherein said dye has a net charge that will maintain the overall net charge of the oligopeptide upon being bound to said oligopeptide.

2. The isoelectric point marker of claim 1, wherein said oligopeptide comprises at least one lysine residue.

3. The isoelectric point marker of claim 2, wherein said oligopeptide comprises a carboxy-terminal lysine residue.

4. The isoelectric point marker of claim 1, wherein said oligopeptide is selected from the group consisting of:

| | |
|---|---|
| No. | Sequence |
| | | |
|---|---|---|
| i) | H₂N-Asp-Gly-Asp-Gly-Lys-COOH ------------------------------- | SEQ ID No. 1 |
| ii) | H₂N-Gly-Asp-Gly-Gly-Asp-Gly-Gly-Lys-COOH ------------------- | SEQ ID No. 2 |
| iii) | H₂N-Gly-GlU-Gly-Gly-Glu-Gly-Glu-Lys-COOH ------------------- | SEQ ID No. 3 |
| iv) | H₂N-Gly-Glu-Glu-Gly-Gly-Lys-COOH --------------------------- | SEQ ID No. 4 |
| v) | H₂N-Gly-Gly-Gly-Gly-Glu-Glu-Lys-COOH ----------------------- | SEQ ID No. 5 |
| vi) | H₂N-Glu-Gly-Asp-Gly-Lys-COOH ------------------------------- | SEQ ID No. 6 |
| vii) | H₂N-Glu-Gly-Glu-Gly-Lys-COOH ------------------------------- | SEQ ID No. 7 |
| viii) | H₂N-Gly-Gly-Glu-Gly-Gly-Glu-Gly-Lys-COOH ------------------- | SEQ ID No. 8 |

5. The isoelectric point marker of claim 1, wherein said oligopeptide is selected from the group consisting of:

| | |
|---|---|
| No. | Sequence |
| | | |
|---|---|---|
| ix) | H₂N-Gly-His-Glu-Gly-Glu-Gly-Lys-COOH -------------------- | SEQ ID No. 9 |
| x) | H₂N-Gly-His-Gly-His-Gly-Glu-Gly-Glu-Gly-Lys-COOH -------- | SEQ ID No. 10 |
| xi) | H₂N-Gly-Gly-His-Gly-Gly-Glu-Gly-Lys-COOH ---------------- | SEQ ID No. 11 |
| xii) | H₂N-Met-Gly-Lys-Gly-Glu-Lys-COOH ------------------------ | SEQ ID No. 12 |
| xiii) | H₂N-Gly-Gly-Lys-Gly-Glu-Lys-COOH ------------------------ | SEQ ID No. 13 |
| xiv) | H₂N-Pro-Gly-Lys-Gly-Glu-Lys-COOH ------------------------ | SEQ ID No. 14 |
| xv) | H₂N-Gly-Tyr-Lys-Tyr-Gly-Lys-COOH ------------------------ | SEQ ID No. 15 |
| xvi) | H₂N-Gly-Tyr-Lys-Gly-Lys-COOH ---------------------------- | SEQ ID No. 16 |

6. The isoelectric point marker of claim 1, wherein said fluorescent dye carries a net +1 charge.

7. The isoelectric point marker of claim 1, wherein said fluorescent dye is covalently reactive with and binds to the primary amine of said lysine residue.

8. The isoelectric point marker of claim 1, wherein said fluorescent dyes are dyes selected from the group consisting of cyanine dyes, squaraine dyes and dipyrromethine boron difluoride dyes, or derivatives thereof.

9. The isoelectric point marker of claim 1, wherein said fluorescent dyes are cyanine dyes, or derivatives thereof.

10. The isoelectric point marker of claim 9, wherein said cyanine dyes are Cy2, Cy3 or Cy5 dyes.

11. The isoelectric point marker of claim 9, wherein said fluorescent dye is a cyanine dye having the structure: 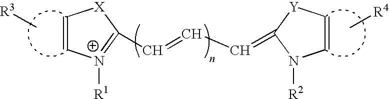
wherein:
the dotted lines each represent carbon atoms necessary for the formation of one or two fused rings having six carbon atoms in each ring;
X and Y are selected from the group consisting of S, O and CH₃—C—CH₃;
n is an integer selected from 1, 2 or 3;
one of R¹ and R² is a group reactive with a nucleophilic group and the remaining R¹ or R² is an alkyl, or is a group containing one or two positive charges; and
R³ and R⁴ are selected from hydrogen and sulfonic acid.

12. The isoelectric point marker of claim 11, wherein X and Y are CH₃—C—CH₃.

13. The isoelectric point marker of claim 11, wherein said fluorescent dye is a cyanine dye having the structure: 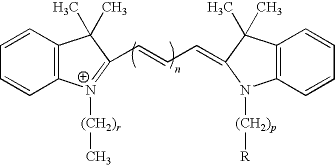
wherein R is selected from the group consisting of isothiocyanate, acid halide, N-hydroxysuccinimidyl ester and N-hydroxysulfosuccinimidyl ester; n is an integer selected from 1, 2 or 3, r is 1 or 2 and p is an integer from 1 to 5.

14. The isoelectric point marker of claim 11, wherein one of R¹ and R² is a reactive group selected from the group consisting of an isothiocyanate, —(CH₂)₅—COOH and —(CH₂)₅—CON— hydroxysuccinimidyl ester; and remaining R¹ or R² is C₁-C₄ alkyl.

15. The isoelectric point marker of claim 9, wherein said fluorescent dye is a cyanine dye having the structure: 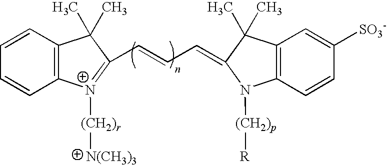
wherein R is selected from the group consisting of isothiocyanate, acid halide, N-hydroxysuccinimidyl ester and N-hydroxysulfosuccinimidyl ester; n is an integer selected from 1, 2 or 3, r is 1 or 2 and p is an integer from 1 to 5.

16. The isoelectric point marker of claim 11, wherein integer n of said dye is 2.

17. The isoelectric point marker of claim 1, further comprising a group controlling molecular weight/migration position without effecting pI.

18. A gel electrophoretic separation method for the analysis of proteins, wherein a set comprising two or more different isoelectric point markers are employed each marker in said set comprising an oligopeptide covalently labelled with a fluorescent dye;
wherein said dye has a net charge that will maintain the overall net charge of the oligopeptide upon being bound to said oligopeptide.

19. The method of claim 18, wherein the isoelectric point pH of each member of the set of isoelectric point markers has a different value and is in the range of from 3 to 11.

20. The method of claim 18, wherein said fluorescent dye is covalently bound to the primary amine of a carboxy-terminal lysine residue in each said isoelectric point marker.

21. The method of claim 18, wherein said set of isoelectric point markers is selected from the group consisting of:

| | |
|---|---|
| No. | Sequence |
| | | |
|---|---|---|
| i) | H₂N-Asp-Gly-Asp-Gly-Lys-COOH ------------------------------- | SEQ ID No. 1 |
| ii) | H₂N-Gly-Asp-Gly-Gly-Asp-Gly-Gly-Lys-COOH ------------------- | SEQ ID No. 2 |
| iii) | H₂N-Gly-GlU-Gly-Gly-Glu-Gly-Glu-Lys-COOH ------------------- | SEQ ID No. 3 |
| iv) | H₂N-Gly-Glu-Glu-Gly-Gly-Lys-COOH --------------------------- | SEQ ID No. 4 |
| v) | H₂N-Gly-Gly-Gly-Gly-Glu-Glu-Lys-COOH ----------------------- | SEQ ID No. 5 |
| vi) | H₂N-Glu-Gly-Asp-Gly-Lys-COOH ------------------------------- | SEQ ID No. 6 |
| vii) | H₂N-Glu-Gly-Glu-Gly-Lys-COOH ------------------------------- | SEQ ID No. 7 |
| viii) | H₂N-Gly-Gly-Glu-Gly-Gly-Glu-Gly-Lys-COOH ------------------- | SEQ ID No. 8 |

22. The method of claim 18, wherein said set of isoelectric point markers is selected from the group consisting of:

| | |
|---|---|
| No. | Sequence |
| | | |
|---|---|---|
| ix) | H₂N-Gly-His-Glu-Gly-Glu-Gly-Lys-COOH -------------------- | SEQ ID No. 9 |
| x) | H₂N-Gly-His-Gly-His-Gly-Glu-Gly-Glu-Gly-Lys-COOH -------- | SEQ ID No. 10 |
| xi) | H₂N-Gly-Gly-His-Gly-Gly-Glu-Gly-Lys-COOH ---------------- | SEQ ID No. 11 |
| xii) | H₂N-Met-Gly-Lys-Gly-Glu-Lys-COOH ------------------------ | SEQ ID No. 12 |
| xiii) | H₂N-Gly-Gly-Lys-Gly-Glu-Lys-COOH ------------------------ | SEQ ID No. 13 |
| xiv) | H₂N-Pro-Gly-Lys-Gly-Glu-Lys-COOH ------------------------ | SEQ ID No. 14 |
| xv) | H₂N-Gly-Tyr-Lys-Tyr-Gly-Lys-COOH ------------------------ | SEQ ID No. 15 |
| xvi) | H₂N-Gly-Tyr-Lys-Gly-Lys-COOH ---------------------------- | SEQ ID No. 16 |


SEQUENCE LISTING (32 sequences)

SEQ ID NO: 1 - protein, 5 aa, Artificial sequence
  Synthetic sequence oligopeptide
DGDGK

SEQ ID NO: 2 - protein, 8 aa, Artificial sequence
  Synthetic sequence oligopeptide
GDGGDGGK

SEQ ID NO: 3 - protein, 8 aa, Artificial sequence
  Synthetic sequence oligopeptide
GEGGEGEK

SEQ ID NO: 4 - protein, 6 aa, Artificial sequence
  Synthetic sequence oligopeptide
GEEGGK

SEQ ID NO: 5 - protein, 7 aa, Artificial sequence
  Synthetic sequence oligopeptide
GGGGEEK

SEQ ID NO: 6 - protein, 5 aa, Artificial sequence
  Synthetic sequence oligopeptide
EGDGK

SEQ ID NO: 7 - protein, 5 aa, Artificial sequence
  Synthetic sequence oligopeptide
EGEGK

SEQ ID NO: 8 - protein, 8 aa, Artificial sequence
  Synthetic sequence oligopeptide
GGEGGEGK

SEQ ID NO: 9 - protein, 7 aa, Artificial sequence
  Synthetic sequence oligopeptide
GHEGEGK

SEQ ID NO: 10 - protein, 10 aa, Artificial sequence
  Synthetic sequence oligopeptide
GHGHGEGEGK

SEQ ID NO: 11 - protein, 8 aa, Artificial sequence
  Synthetic sequence oligopeptide
GGHGGEGK

SEQ ID NO: 12 - protein, 6 aa, Artificial sequence
  Synthetic sequence oligopeptide
MGKGEK

SEQ ID NO: 13 - protein, 6 aa, Artificial sequence
  Synthetic sequence oligopeptide
GGKGEK

SEQ ID NO: 14 - protein, 6 aa, Artificial sequence
  Synthetic sequence oligopeptide
PGKGEK

SEQ ID NO: 15 - protein, 6 aa, Artificial sequence
  Synthetic sequence oligopeptide
GYKYGK

SEQ ID NO: 16 - protein, 5 aa, Artificial sequence
  Synthetic sequence oligopeptide
GYKGK

SEQ ID NO: 17 - protein, 11 aa, Saccharomyces cerevisiae
DCDSLSSLVTR

SEQ ID NO: 18 - protein, 13 aa, Saccharomyces cerevisiae
DTDYVPIVVVGNK

SEQ ID NO: 19 - protein, 13 aa, Saccharomyces cerevisiae
DSDVTWLYGPIVR

SEQ ID NO: 20 - protein, 13 aa, Saccharomyces cerevisiae
DLDLPTQQILVAR

SEQ ID NO: 21 - protein, 12 aa, Saccharomyces cerevisiae
DLDAVNGSNGSK

SEQ ID NO: 22 - protein, 12 aa, Saccharomyces cerevisiae
DQDSAVVSSNIK

SEQ ID NO: 23 - protein, 13 aa, Saccharomyces cerevisiae
DTDMVLIPAGVPR

SEQ ID NO: 24 - protein, 11 aa, Saccharomyces cerevisiae
DIDILVNNAGK

SEQ ID NO: 25 - protein, 10 aa, Saccharomyces cerevisiae
DGDYNLVGSK

SEQ ID NO: 26 - protein, 13 aa, Saccharomyces cerevisiae
DIDNGYTLSLMYK

SEQ ID NO: 27 - protein, 12 aa, Saccharomyces cerevisiae
DGDQVVFMVSQK

SEQ ID NO: 28 - protein, 10 aa, Saccharomyces cerevisiae
DNDVQLAATK

SEQ ID NO: 29 - protein, 9 aa, Saccharomyces cerevisiae
DVDLVNAAR

SEQ ID NO: 30 - protein, 12 aa, Saccharomyces cerevisiae
DADLLNSVVVQR

SEQ ID NO: 31 - protein, 10 aa, Saccharomyces cerevisiae
DLDAAQGTLK

SEQ ID NO: 32 - protein, 13 aa, Saccharomyces cerevisiae
DSDYIPVVVVGNK